(19) United States
(12) Patent Application Publication
(10) Pub. No.: US 2025/0195592 A1
(43) Pub. Date: Jun. 19, 2025

(54) METHODS FOR IMPROVING SLEEP QUALITY, INCREASING MITOCHONDRIAL EXPRESSION, PROMOTING CELL REPAIRING, OR FIRMING SKIN BY USING SPARASSIA CRISPA EXTRACT

(71) Applicant: TCI CO., LTD., Taipei (TW)

(72) Inventors: YUNG-HSIANG LIN, Taipei (TW); FAN-JHEN DAI, Taipei (TW)

(21) Appl. No.: 18/409,817

(22) Filed: Jan. 11, 2024

(30) Foreign Application Priority Data
Dec. 14, 2023 (TW) 112148877

(51) Int. Cl. A61K 36/07; A61K 8/9728; A61P 25/00; A61Q 19/08

(52) U.S. Cl. CPC A61K 36/07; A61K 8/9728; A61P 25/00; A61Q 19/08; A61K 2236/331

(57) ABSTRACT

Methods for improving sleep quality, increasing mitochondrial expression, repairing cell damage, and firming skin of a subject in need thereof are provided. The methods include: administering to the subject an effective dose of Sparassia crispa extract. The Sparassia crispa extract is obtained by extracting Sparassia crispa by water. The Sparassia crispa extract has at least one of the following effects: increasing content of gamma-aminobutyric acid (GABA), contributing to nerve relaxation, enhancing activity of mitochondria in cells, extending cell lifespan, regulating autophagy of mitochondria in cells, regulating ATG1 gene, ATG8 gene, CCT5 gene, CCT7 gene, Pink1 gene, Parkin gene, IRT1 gene, FOXO gene, PARP1 gene, and SOD3 gene in cells, reducing laxity of the skin of the subject, and reducing dryness of the skin of the subject.

### CROSS-REFERENCE TO RELATED APPLICATION

This non-provisional application claims priority under 35 U.S.C. § 119(a) to Patent Application No. 112148877 filed in Taiwan, R.O.C. on Dec. 14, 2023, the entire contents of which are hereby incorporated by reference.

### REFERENCE OF ELECTRONIC SEQUENCE LISTING

The contents of the electronic sequence listing (P223413USI.xml; Size: 18,865 bytes; and Date of Creation: Jan. 8, 2024) is herein incorporated by reference in its entirety.

### BACKGROUND

### Technical Field

The present disclosure relates to a Sparassia crispa extract, and particularly relates to a method of using the Sparassia crispa extract, where the Sparassia crispa extract has the effects of improving sleep quality, improving mitochondrial expression, repairing cell damage, or firming skin of a subject.

### Related Art

Most of common mushrooms grow in the dark, while Sparassia crispa needs more than ten hours of sunlight every day to grow. Therefore, it is currently the only known “sunshine mushroom”. The fruiting body of the Sparassia crispa is crisp and tender, and has a fragrant and unique taste. Therefore, it is a delicacy recognized by gourmets.

There are many causes of insomnia, which may be related to stress, emotions, lifestyle habits, diseases, medication, brain degeneration or the like. Insomnia not only harms physical and mental health, but also will increase the risk of suffering from various diseases. However, sleeping pills may cause dependence, tolerance, and side effects, a comfortable sleep therapy may lead to severe anesthesia reactions, while sleep appliances may cause allergies or breathing difficulties.

### SUMMARY

The growth environment of Sparassia crispa is different from that of common mushrooms, which results in that the Sparassia crispa has different active ingredients. In view of this, in order to develop applications of the Sparassia crispa on more levels, use of a Sparassia crispa extract for preparing a composition for improving sleep quality, improving mitochondrial expression, repairing cell damage, or firming skin of a recipient is provided.

In some embodiments, the present disclosure provides a method for reducing light sleep and/or dreaming in a subject in need thereof. The method includes: administering to the subject an effective dose of the Sparassia crispa extract, where the Sparassia crispa extract is obtained by extracting Sparassia crispa by water.

In some embodiments, the Sparassia crispa extract increases gamma-aminobutyric acid (GABA) content.

In some embodiments, the Sparassia crispa extract contributes to nerve relaxation of the subject.

In some embodiments, the present disclosure provides a method for improving mitochondrial expression in a subject in need thereof. The method includes: administering to the subject an effective dose of the Sparassia crispa extract, where the Sparassia crispa extract is obtained by extracting Sparassia crispa by water.

In some embodiments, the Sparassia crispa extract enhances activity of mitochondria in cells of the subject.

In some embodiments, the Sparassia crispa extract extends cell lifespan of the subject.

In some embodiments, the Sparassia crispa extract regulates autophagy of mitochondria in cells of the subject.

In some embodiments, the Sparassia crispa extract regulates an expression level of a mitochondrial expression-related gene in cells of the subject, and the mitochondrial expression-related gene is at least one of: a CCT5 gene, a CCT7 gene, a Pink1 gene, a Parkin gene, an SIRT1 gene, an FOXO gene, a PARP1 gene, and an SOD3 gene.

In some embodiments, the present disclosure provides a method for repairing cell damage in a subject in need thereof. The method includes: administering to the subject an effective dose of the Sparassia crispa extract, where the Sparassia crispa extract is obtained by extracting Sparassia crispa by water.

In some embodiments, the Sparassia crispa extract regulates autophagy of cells of the subject.

In some embodiments, the Sparassia crispa extract enhances an expression level of an ATG1 gene and/or an ATG8 gene of the subject.

In some embodiments, the Sparassia crispa extract increases gamma-aminobutyric acid content of the subject.

In some embodiments, the present disclosure provides a method for firming skin in a subject in need thereof. The method includes: administering to the subject an effective dose of the Sparassia crispa extract, where the Sparassia crispa extract is obtained by extracting Sparassia crispa by water.

In some embodiments, the Sparassia crispa extract reduces laxity of the skin of the subject.

In some embodiments, the Sparassia crispa extract reduces dryness of the skin of the subject.

In some embodiments, the Sparassia crispa extract is extracted from a fruiting body of Sparassia crispa.

In some embodiments, the Sparassia crispa extract is obtained by extracting Sparassia crispa in water at 90° C.±5° C. for 60 min.

In summary, the Sparassia crispa extract of the embodiments of the present disclosure can be used for preparing a composition for improving sleep quality, increasing mitochondrial expression, repairing cell damage, or firming the skin of a subject. In some embodiments, the Sparassia crispa extract has at least one of the following effects: increasing gamma-aminobutyric acid (GABA) content, contributing to nerve relaxation, enhancing activity of mitochondria in cells, extending cell lifespan, regulating autophagy of mitochondria in cells, regulating ATG1 gene, ATG8 gene, CCT5 gene, CCT7 gene, Pink1 gene, Parkin gene, SIRT1 gene, FOXO gene, PARP1 gene, and SOD3 gene in cells, reducing laxity of the skin of the subject, and reducing dryness of the skin of the subject.

### BRIEF DESCRIPTION OF THE DRAWINGS

FIG. 1 is a bar chart showing the test results of a Sparassia crispa extract in promoting GABA content according to an embodiment of the present disclosure;

FIG. 2 is a bar chart showing the test results of a Sparassia crispa extract in regulating gene expression according to an embodiment of the present disclosure;

FIG. 3 is a bar chart showing the test results of a Sparassia crispa extract in regulating gene expression according to an embodiment of the present disclosure;

FIG. 4 is a bar chart showing the test results of a Sparassia crispa extract in regulating gene expression according to an embodiment of the present disclosure;

FIG. 5 is a bar chart showing the test results of a Sparassia crispa extract in regulating gene expression according to an embodiment of the present disclosure,

FIG. 6 is a bar chart showing the test results of a Sparassia crispa extract in regulating gene expression according to an embodiment of the present disclosure;

FIG. 7 is a bar chart showing the test result of average skin laxity in human experiments according to an embodiment of the present disclosure;

FIG. 8 is a bar chart showing the result of average GABA content in blood in human experiments according to an embodiment of the present disclosure;

FIG. 9 is a bar chart showing the statistical result of questionnaire for human experiments according to an embodiment of the present disclosure; and

FIG. 10 is a bar chart showing the statistical result of questionnaire for human experiments according to an embodiment of the present disclosure.

### DETAILED DESCRIPTION

Sparassia crispa as used herein refers to a fruiting body of the Sparassia crispa. The fruiting body has a milky white to yellow appearance, and presents a flat and curly leaf-like structure.

In some embodiments, Sparassia crispa used for extraction of a Sparassia crispa extract may be fresh, dried, or frozen. In some embodiments, drying may be air drying, sun drying, drying in shade, or freeze drying.

In some embodiments, Sparassia crispa used for extraction of a Sparassia crispa extract may be intact Sparassia crispa or Sparassia crispa obtained after physical processing procedures such as chopping, dicing, slicing, milling, grinding, or otherwise that alter the size and physical integrity of raw materials.

In some embodiments, the Sparassia crispa extract is a liquid extract prepared by a water extraction step. In the water extraction step, extraction is performed at a specific temperature for a specific time using water as a solvent to prepare the Sparassia crispa liquid extract. In some embodiments, the specific temperature refers to a temperature between 95° C. and 85° C. In some embodiments, the specific temperature is 90° C.±5° C. In some embodiments, the specific temperature is 90° C. In some embodiments, the specific time refers to 60 min to 80 min. In some embodiments, the specific time refers to 60 min.

In some embodiments, the water extraction step further includes measuring Degrees Brix (°Bx) of the Sparassia crispa liquid extract after a specific time, and preparing the Sparassia crispa liquid extract when the Degrees Brix reaches 2.9°Bx±0.5°Bx. In some embodiments, the water extraction step further includes measuring Degrees Brix of the Sparassia crispa liquid extract after a specific time, and extending the specific time for another 30 min when the measured Degrees Brix does not reach 2.4°Bx. This means that the Sparassia crispa liquid extract is prepared with the Degrees Brix of 2.9°Bx±0.5°Bx as an acceptance standard. In some embodiments, the Sparassia crispa liquid extract is a Sparassia crispa extract.

In some embodiments, in the water extraction step, the weight ratio of the water to the Sparassia crispa is (15 to 5):1. By way of examples, the weight ratio of the water to the Sparassia crispa is 10:1. Here, if there is too little solvent or the specific time is too short, the extraction efficiency will significantly decrease. If the specific time is too long, active ingredients in the extract may be degraded.

In some embodiments, the Sparassia crispa extract is a Sparassia crispa liquid extract prepared by a water extraction step and a filtration step. The filtration step refers to a step of filtering the Sparassia crispa extract after the water extraction step through a sieve or centrifugation to remove solid matter in the Sparassia crispa extract. By way of examples, the Sparassia crispa extract is filtered through a 400-mesh sieve to prepare the Sparassia crispa liquid extract.

In some embodiments, the Sparassia crispa extract is a Sparassia crispa liquid extract prepared by a water extraction step, a filtration step and a concentration step. The concentration step refers to the step of removing excess water from the Sparassia crispa extract after the water extraction step or water extraction step and the filtration step to reduce the storage volume of the Sparassia crispa extract. By way of examples, concentration under reduced pressure can be performed at 60° C.±5° C. using a concentrator (brand/model: BUCHI-Rotavapor R-100), and is stopped until Degrees Brix of a solution is 10±0.5 to prepare a Sparassia crispa liquid extract. In some other embodiments, the concentration under reduced pressure can be performed at 50° C. to 82° C.

In some embodiments, the Sparassia crispa extract is used for preparing a composition for reducing light sleep and/or dreaming. In some embodiments, the Sparassia crispa extract is used for increasing gamma-aminobutyric acid (GABA) content. In some embodiments, the Sparassia crispa extract contributes to nerve relaxation.

In some embodiments, the present disclosure provides use of a Sparassia crispa extract, which is used for preparing a composition for increasing mitochondrial expression, where the Sparassia crispa extract is extracted from Sparassia crispa using water as a solvent. In some embodiments, the Sparassia crispa extract is used for enhancing activity of mitochondria in a cell. In some embodiments, the Sparassia crispa extract is used for extending a cell lifespan. In some embodiments, the Sparassia crispa extract is used for regulating autophagy of mitochondria in a cell. In some embodiments, the Sparassia crispa extract is used for regulating an expression level of a mitochondrial expression-related gene in human cells, and the mitochondrial expression-related gene is at least one of: a CCT5 gene, a CCT7 gene, a Pink1 gene, a Parkin gene, an SIRT1 gene, an FOXO gene, a PARP1 gene, and an SOD3 gene.

In some embodiments, the Sparassia crispa extract is used for preparing a composition for repairing cell damage, where the Sparassia crispa extract is extracted from Sparassia crispa using water as a solvent. In some embodiments, the Sparassia crispa extract is used for regulating autophagy of a cell. In some embodiments, the Sparassia crispa extract is used for enhancing an expression level of an ATG1 gene and/or an ATG8 gene.

In some embodiments, the Sparassia crispa extract is used preparing a composition for firming skin of a subject. In some embodiments, the Sparassia crispa extract is used for reducing laxity of the skin of the subject. In some embodiments, the Sparassia crispa extract is used for reducing dryness of the skin of the subject.

In some embodiments, the Sparassia crispa extract is used for enhancing activity of mitochondria in skin cells. Here, the mitochondria are an energy manufacturing factory of cells. The activity of the mitochondria will be reduced by the fact that the mitochondria are damaged by an external environment or degrade naturally, while diabetes, heart disease, arthritis and the like are also found to be related to a variation of mitochondrial DNA. For example, if the skin is irradiated by UV light for a long time, the mitochondrial DNA will mutate to reduce the activity of the mitochondria. If the activity of the mitochondria can be promoted, the metabolic rate and growth rate of the cell can be increased, and then the skin is repaired. From external observation, it can be seen that skin aging is slowed down. Furthermore, there are a large number of mitochondria in the brain. If the function of the mitochondria can be maintained normally, the smooth operation of the brain can be maintained, thereby avoiding insomnia caused by brain degeneration.

In some embodiments, the Sparassia crispa extract is used for regulating an expression level of a mitochondria-related gene to improve mitochondrial expression. In some embodiments, the mitochondria-related gene is at least one of: a CCT5 gene (Gene ID: 22948), a CCT7 gene (Gene ID: 10574), a Pink1 gene (Gene ID: 65018), a Parkin gene (Gene ID: 5071), an SIRT1 gene (Gene ID: 23411), an FOXO gene (Gene ID: 2308), a PARP1 gene, and an SOD3 gene (Gene ID: 6649).

In some embodiments, the Sparassia crispa extract is used for regulating an expression level of a cell autophagy-related gene to repair cell damage. In some embodiments, the cell autophagy-related gene is at least one of an Atg1 gene (Gene ID: 8408) and an Atg8 gene (Gene ID: 11345).

The CCT5 gene and the CCT7 gene regulate a mitochondrial activity-related gene. The Pink1 gene and the Parkin gene regulate a mitochondrial expression and degradation-related gene. This means that an energy source of a cell can be activated and aging of the cell can be delayed by promoting the expression levels of the CCT5 gene, the CCT7 gene, the Pink1 gene, and the Parkin gene.

The ATG1 gene and the ATG8 gene are involved in the autophagy of the cell, and metabolizing old waste organelles, and recycling recyclable substances by regulating the autophagy of the cell. This means that cell repairing capacity can be enhanced by promoting the expression levels of the ATG1 gene and the ATG8 gene.

When the PARP1 gene is expressed, NAD⁺ will be inhibited, thereby reducing mitochondrial activity, and influencing cellular energy. This means that the mitochondrial activity can be enhanced by inhibiting the expression level of the PARP1 gene. The SIRT1 (sirtuin) gene is to enhance the capacity of the cell to balance oxidative stress by promoting the mitochondrial activity. The FOXO gene is regulated by the SIRT1 gene, which in turn controls the expression of SOD3 gene. The SOD3 gene is regulated by the FOXO gene, which in turn influences extension of a cell lifespan.

In some embodiments, the aforementioned composition may be a health-care product or a health-care food. In some embodiments, the aforementioned composition is a health-care product or health-care food for non-medical purposes, in other words, the health-care product or the health-care food includes an effective amount of Sparassia crispa extract.

In some embodiments, the aforementioned health-care composition can be manufactured into a dosage form suitable for being intestinally or orally administrated by using a technology well known to those skilled in this art. These dosage forms include, but are not limited to, a tablet, a troche, a lozenge, a pill, a capsule, dispersible powder or a granule, a solution, a suspension, an emulsion, syrup, an elixir, slurry and the like.

In some embodiments, the aforementioned health-care composition can be manufactured into a dosage form suitable for being parenterally or topically administrated by using a technology well known to those skilled in this art. These dosage forms include, but are not limited to: an injection, sterile powder, an external preparation and the like. In some embodiments, the health-care composition can be administered by parenteral routes selected from the group consisting of subcutaneous injection, intradermal injection, intradermal injection and intralesional injection.

In some embodiments, the health-care composition may further include a pharmaceutically acceptable carrier widely used in a food manufacturing technology. For example, the pharmaceutically acceptable carrier may include one or more of the following reagents: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome and the like. Selection and number regarding these reagents fall within the scope of the professional quality and routine technology well known to those skilled in the art.

In some embodiments, the pharmaceutically acceptable carrier includes a solvent selected from the group consisting of water, normal saline, phosphate buffered saline (PBS) and an aqueous solution containing alcohol.

In some embodiments, the aforementioned health-care composition may be an edible composition. In some embodiments, the edible composition can be manufactured into a food product or may be a food additive, which means that the food additive is added by a well-known method in food material preparation to prepare the food product, or added in the manufacturing process of the food product. Here, the food product may be formulated with edible materials into a product for human or animal consumption.

In some embodiments, the food product may be, but is not limited to, beverages, fermented foods, bakery products, health foods and dietary supplements.

### Example 1: Preparation of Sparassia crispa Extract

Firstly, dried fruiting bodies of Sparassia crispa produced in a high-altitude fir forest zone in China were used as a raw material.

Next, a water extraction step was carried out. Using water as a solvent, the water was heated to 90° C.±5° C. (i.e., specific temperature), and then, the raw material of the Sparassia crispa was added. The Sparassia crispa raw material and the water were mixed in a weight ratio of 1:10. This means that after the Sparassia crispa was mixed with the water, extraction was performed at 90° C.±5° C. for 60 min (i.e., specific time) to obtain a Sparassia crispa liquid extract.

Subsequently, a filtration step was carried out. After being cooled to room temperature, the Sparassia crispa liquid extract was sieved through a 400-mesh sieve, and then its filtrate was taken.

Afterwards, a concentration step was carried out. The above-mentioned filtrate was taken and was subjected to concentration under reduced pressure at a set temperature of 60° C.±5° C. using a concentrator (brand/model: BUCHI-Rotavapor R-100), and the concentration was stopped until Degrees Brix of a solution was 10±0.5 to obtain a concentrate, i.e., a Sparassia crispa extract.

### Example 2: Test of Sparassia crispa Extract to Promote Secretion of GABA

GABA has a full name of gamma-aminobutyric acid. When we enter deep sleep, GABA content of brain nerve cells will be increased. Stabilization of the GABA content of the brain also helps fight depression and help sleep. However, due to a blood-brain barrier, the efficiency by direct consumption of GABA is very low. If self-synthesis of a nerve cell can be promoted, the efficiency cannot be influenced by the blood-brain barrier.

Meanwhile, GABA can regulate autonomic nerves, alleviate tension, reduce stress, and improve sleep quality, and also has the functions of promoting secretion of a growth hormone, enhancing skeletal muscles, enhancing immunity, reducing fat, and the like. In this test, human brain nerve cells SH-SY5Y were used, and then the content of GABA secreted by the cells was detected using a GABA ELISA Kit. Whether the Sparassia crispa extract could help increase the secretion of GABA of the brain nerve cells or not was evaluated.

### Material and Device Description:

Cell line: Human neuroblastoma cells (SH-SY5Y), taken from the American Type Culture Collection (ATCC), with a deposit number of Cat. CRL-2266, hereinafter referred to as nerve cells.

Cell medium: A minimum essential medium (Dulbecco's modified Eagle medium (hereinafter referred to DMEM)) (Gibco, with a product number of 12100-046) supplemented with 10% of fetal bovine serum (FBS; Thermo, with a product number of 10437-028), and 1% of penicillin-streptomycin (Thermo, Cat. 15240062).

A ELISA Kit for GammaAminobutyric Acid (gABA) (CEA900Ge) contains GABA.

Solvent: 1×DPBS (Gibco, Cat. 14200-075), a cell lysis buffer (Thermo, Cat. FNN0011).

### Test Flow:

Firstly, the nerve cells were implanted into a 24-well cell culture plate at a density of 1×10⁴ nerve cells, each well containing 0.5 ml of cell medium, and cultured in a carbon dioxide incubator at 37° C. for 24 h.

The nerve cells were divided into an experimental group and a blank group. The cell medium in each group was removed and replaced with a 500 μl/well test medium, then the test medium was placed at 37° C. and further cultured for 24 h.

The test medium in the experimental group was a cell medium containing 0.0625% of Sparassia crispa extract obtained in Example 1.

The test medium in the blank group was a pure cell medium.

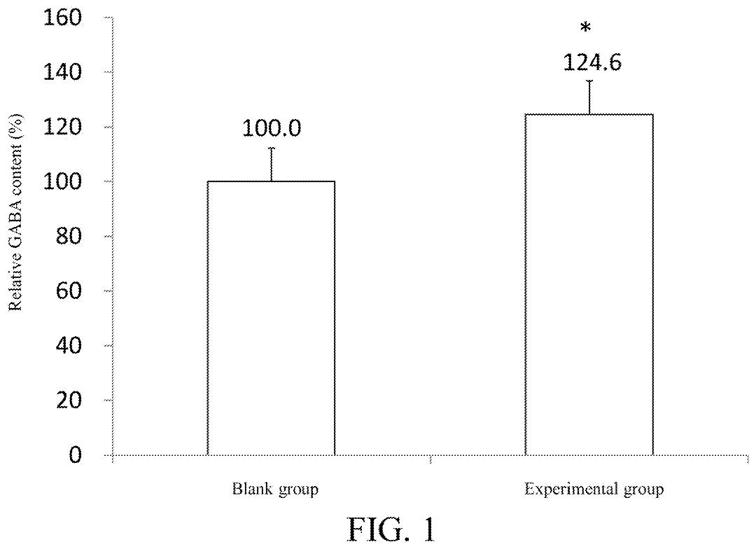

Supernatant in each group was removed, and then the culture plate was washed twice with 1×DPBS and the cell lysis buffer was added to lyse the cells. Centrifugation was performed at 4° C. at 13,000 rpm for 5 min, and the supernatant was collected and stored in a 1.5 mL microcentrifuge tube. Treatment was performed with the detection kit for gamma-aminobutyric acid to obtain the GABA content in each group. The test results are as shown in FIG. 1.

The GABA content shown in the figure is presented in relative rate, where a standard deviation was calculated using an STDEV formula of Excel software, and whether there was a statistically significant difference or not was analyzed with a single-tailed student t-test in an Excel software. In the figure, “*” represents that the p value is less than 0.05, and “**” represents that the p value is less than 0.01. The more the “*”, the more significant the statistical differences.

See FIG. 1. Values in the blank group were regarded as 100%. Compared with that of the blank group, the GABA content in the experimental group was 124.6%, and there was a statistically significant difference between the experimental group and the blank group. In other words, the Sparassia crispa extract could significantly promote the secretion of GABA by the human nerve cells. It can be seen from this that the Sparassia crispa extract can improve sleep quality in humans.

### Example 3: Test of Sparassia crispa Extract on Expression of Mitochondria-Related Genes

### Materials and Instruments:

Cell line: Human skin fibroblasts CCD-966Sk, taken from the American Type Culture Collection (ATCC), with a deposit number of Cat. CRL-1881, hereinafter referred to as skin fibroblasts.

Cell medium: Formulated with 90% of minimum essential medium (Eagle) in Earle's BSS supplemented with ingredients to contain 0.1 mM non-essential amino acid solution, 1.5 g/L sodium bicarbonate, 1 mM sodium pyruvate, and 10% fetal bovine serum (hereinafter referred to as FBS) (purchased from Gibco).

RNA extraction reagent kit, purchased from Genemark.

SuperScript® III reverse transcriptase, purchased from Invitrogen.

ABI StepOnePlus™ Real-Time PCR system, purchased from Thermo Fisher Scientific.

KAPA SYBR FAST qPCR Master Mix (2×) Kit, purchased from KAPA Biosystems, with a product number of KK4600.

### Test Flow:

The skin fibroblasts were inoculated into 6-well culture plate containing 2 mL of cell medium per well at a density of 1×10⁵ skin fibroblasts per well, and cultured at 37° C. for 48 h.

After the culture, the skin fibroblasts were divided into several blank groups and experimental groups for comparison of expression of different genes. A culture solution in the blank group did not contain any extracts, while a culture solution in the experimental group contained 0.0625% of Sparassia crispa extract prepared in Example 1. Each group was subjected to three replicates, and cultured at 37° C. for 24 h.

The culture solutions in the cultured blank group and experimental group were removed, and rinsing was performed with PBS.

After the rinsing, a cell membrane of a HPEK-50 cell in each group was broken open with a cell lysis buffer in the RNA extraction reagent kit to form a cell solution.

RNA in the cell solution in each group was extracted using the RNA extraction reagent kit.

2000 nanograms (ng) of the RNA extracted was taken from each group to serve as a template, and the extracted RNA was reverse-transcribed by the SuperScript® III reverse transcriptase into corresponding cDNA.

A quantitative real-time reverse transcription polymerase chain reaction was performed on the cDNA by using the ABI StepOnePlus™ Real-Time PCR system, with the KAPA SYBR FASTqPCR Master Mix (2×) Kit and a combined primer of Table 1, to observe expression levels of various target genes of the HPEK-50 cells in the blank group and the experimental group and melting curves thereof. Instrument set conditions for the quantitative real-time reverse transcription polymerase chain reaction were: reaction at 95° C. for 20 s, reaction at 95° C. for 3 s, and reaction at 60° C. for 30 s, which was repeated for 40 cycles.

Relative expression levels of target genes were determined by using a 2^{-ΔΔCt} method. The so-called relative expression level was defined as a fold change of an RNA expression level of the target gene in the experimental group relative to that of the same gene in the blank group. In the 2^{-ΔΔCt} method, the fold change was calculated with a cycle threshold of a TBP gene as a cycle threshold (Ct) of a reference gene for internal control according to the following formula:

$$\begin{matrix}Δ Ct = C {t}_{target gene of experimental group / target gene of blank group} - {Ct}_{GAPDH} \\ ΔΔ Ct = Δ {Ct}_{target gene of experimental group} - Δ {Ct}_{target gene of blank group} \\ Fold change = {2}^{- ΔΔ Ctmean}\end{matrix}$$

TABLE 1

| Primer name | Sequence number | Sequence |
|---|---|---|
| CCT5-F | SEQ ID NO: 1 | CGGATAAGTGCCCCACCTTA |
| CCT5-R | SEQ ID NO: 2 | TCCAGTGCGTCGGCAAA |
| CCT7-F | SEQ ID NO: 3 | GTGGCATGGACAAGCTTATTGTAG |
| CCT7-R | SEQ ID NO: 4 | CAGAATTGTGGCCCCATCA |
| Pink1-F | SEQ ID NO: 5 | CTGTGGTGGCTAGTGCTCCT |
| Pink1-R | SEQ ID NO: 6 | TCCAGACGTGAGACAGTTGG |
| Parkin-F | SEQ ID NO: 7 | GCAGAGACCGTGGAGAAAAG |
| Parkin-R | SEQ ID NO: 8 | CTTTTCTCCACGGTCTCTGC |
| Atg1-F | SEQ ID NO: 9 | CAGGAGGACGAGAACACGGTGTC |
| Atg1-R | SEQ ID NO: 10 | GGAAGGTTCTTTGGCACCAGCAC |
| Atg8-F | SEQ ID NO: 11 | TATCCAGACCGTGTGCCCGTC |
| Atg8-R | SEQ ID NO: 12 | GTGGATGCGCTTGCGAATGAGG |
| SIRT1-F | SEQ ID NO: 13 | TAGCCTTGTCAGATAAGGAAGGA |
| SIRT1-R | SEQ ID NO: 14 | ACAGCTTCACAGTCAACTTTGT |
| FOXO-F | SEQ ID NO: 15 | CGGACAAACGGCTCACTCT |
| FOXO-R | SEQ ID NO: 16 | GGACCCGCATGAATCGACTAT |
| PARP1-F | SEQ ID NO: 17 | AGCGTGTTTCTAGGTCGTGG |
| PARP1-R | SEQ ID NO: 18 | CATCAAACATGGGCGACTGC |
| SOD3-F | SEQ ID NO: 19 | AGCTGGAAAGGTGCCCGA |
| SOD3-R | SEQ ID NO: 20 | CTTGGCGTACATGTCTCGGAT |

Here, statistically significant differences between measurement results of the blank group and the experimental group were obtained through statistical analysis of a student t-test. (In the figure, “*” represents that its p value is less than 0.05 in comparison with that in the blank group, “**” represents that its p value is less than 0.01 in comparison with that in the blank group, and “***” represents that its p value is less than 0.001 in comparison with that in the blank group. The more the “*”, the more significant the statistical difference).

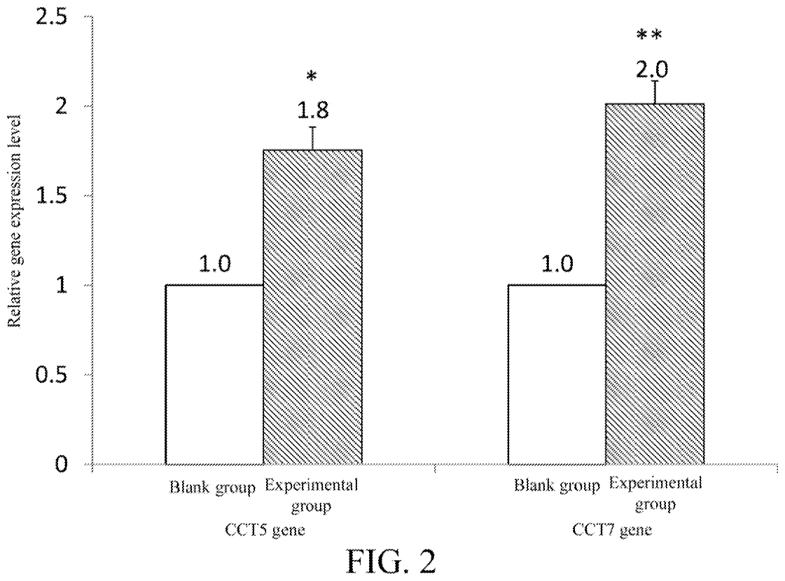

See FIG. 2 for the test results. The skin fibroblasts in the blank group were not subjected to any treatment, which means that expression levels of their gene were set as 1.0 under normal physiological metabolism conditions. An expression level of CCT5 gene in a CCT5 gene experimental group was 1.8 times that of the blank group, while an expression level of CCT7 gene in a CCT7 gene experimental group was 2 times that of the blank group. It can be seen that the expression levels of the CCT5 gene and the CCT7 gene can be significantly increased after treatment with the Sparassia crispa extract, thereby enhancing mitochondrial activity.

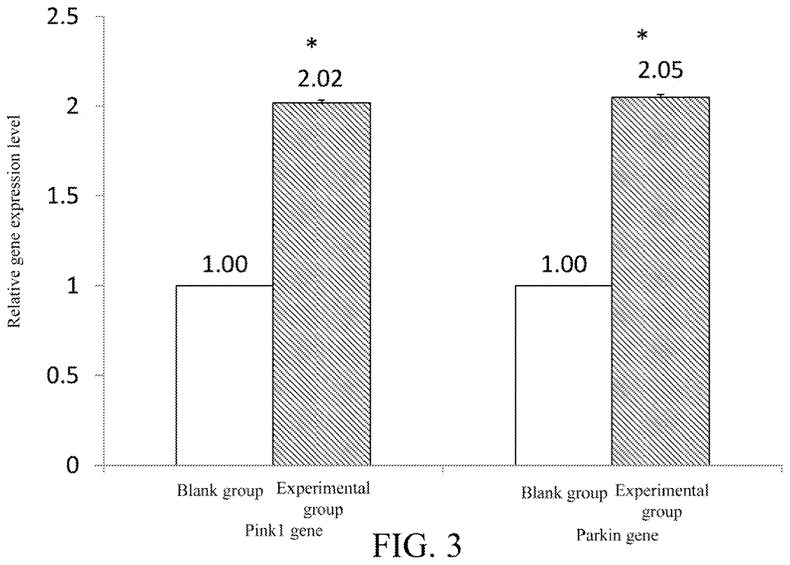

See FIG. 3 for the test results. The skin fibroblasts in the blank group were not subjected to any treatment, which means that expression levels of their gene were set as 1.0 under normal physiological metabolism conditions. An expression level of Pink1 gene in the Pink1 gene experimental group was 2.02 times that of the blank group, while an expression level of Parkin gene in the Parkin gene experimental group was 2.05 times that of the blank group. It can be seen that the expression levels of the Pink1 gene and the Parkin gene can be significantly increased after treatment with the Sparassia crispa extract, thereby activating an energy source of the cell, and delaying aging of the cell.

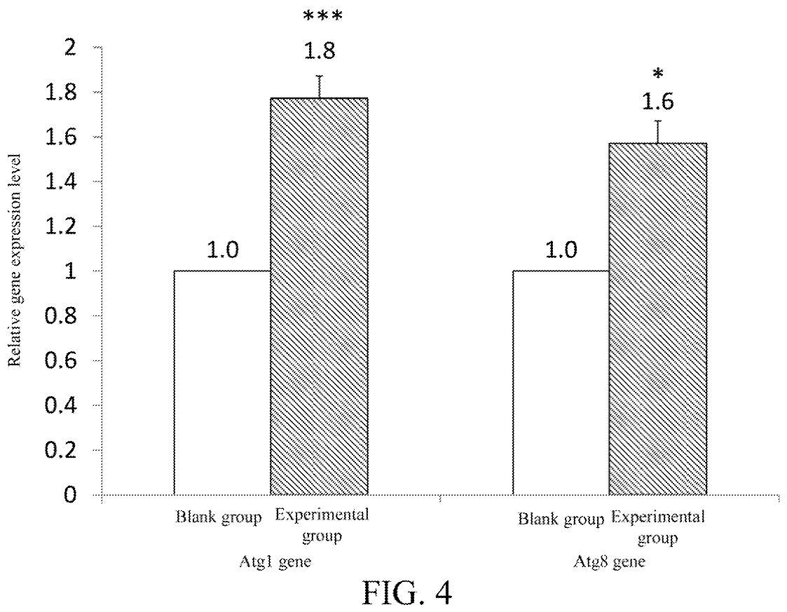

See FIG. 4 for the test results. The skin fibroblasts in the blank group were not subjected to any treatment, which means that expression levels of their gene were set as 1.0 under normal physiological metabolism conditions. An expression level of Atg1 gene in the Atg1 gene experimental group was 1.8 times that of the blank group, while an expression level of Atg8 gene in the Atg8 gene experimental group was 1.6 times that of the blank group. It can be seen that the expression levels of the Atg1 gene and the Atg8 gene can be significantly increased after treatment with the Sparassia crispa extract, thereby improving cell repairing capacity.

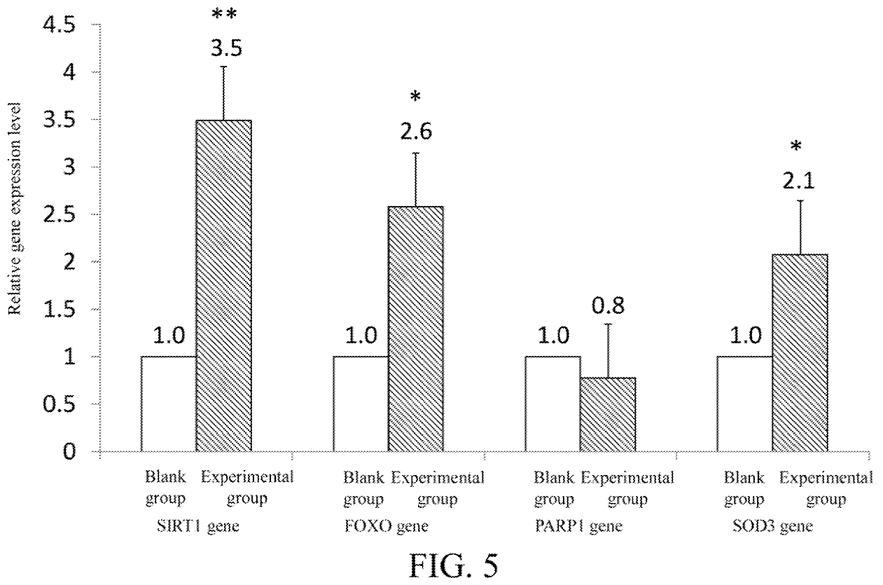

See FIG. 5 for the test results. The skin fibroblasts in the blank group were not subjected to any treatment, which means that expression levels of their gene were set as 1.0 under normal physiological metabolism conditions. An expression level of SIRT1 gene in the SIRT1 gene experimental group was 3.5 times that of the blank group, an expression level of FOXO gene in the FOXO gene experimental group was 2.6 times that of the blank group, an expression level of PARP1 gene in the PARP1 gene experimental group was 0.8 times that of the blank group, while an expression level of SOD3 gene in the SOD3 gene experimental group was 2.1 times that of the blank group. It can be seen that the expression levels of the SIRT1 gene, the FOXO gene and the SOD3 gene can be significantly increased after treatment with the Sparassia crispa extract, thereby extending a cell lifespan, while the expression level of the PARP1 gene is inhibited, thereby enhancing the mitochondrial activity.

It can be seen from this that the Sparassia crispa extract can significantly enhance the expression levels of the ATG1 gene, the ATG8 gene, the CCT5 gene, the CCT7 gene, the Pink1 gene, the Parkin gene, the SIRT1 gene, the FOXO gene, and the SOD3 gene, and can inhibit the expression level of the PARP1 gene.

### Example 4: Human Body Test of Sparassia crispa Extract

Subject: 8 subjects. Each subject was an adult aged 20 or above.

Test items: Skin laxity, expression levels of SIRT1 gene, OXO gene, and SOD3 gene in blood, GABA content in blood, skin questionnaire survey, and sleep questionnaire survey.

Regarding the skin laxity, the facial skin of the same subject before and after consumption was detected using a skin elasticity detection probe Cutometer® MPA580 (C+K Multi Probe Adapter System, Germany) purchased from the Courage+Khazaka electronic in Germany. The test principle was based on principles of suction and stretching. Negative pressure was generated on the surface of tested skin to suck the skin into a test probe. The depth of the skin sucked into the probe was detected with an optical test system, and the laxity was analyzed and calculated with software.

Human peripheral blood mononuclear cells (PBMCs) were isolated from the blood of the subject, and the expression levels of the SIRT1 gene, the FOXO gene, and the SOD3 gene were measured.

The GABA content in the blood was detected by Dajiang Gene.

Regarding the skin questionnaire survey, self-assessment was conducted on the two items: the skin laxity and skin dryness, and an option was made from the following five options: none, mild, obvious, severe, and very severe. Then the option was converted into a score for expression, where 1 point was for none, 2 points were for mild, 3 points were for obvious, 4 points were for severe, and 5 points were for very severe. A score before taking was then subtracted from a score after taking, and then the difference was divided by the score before taking, and the quotient was converted into a percentage.

Regarding the sleep questionnaire survey, self-assessment was conducted on the two items: level of light sleep and level of dreaming, and an option was made from the following five options: none, mild, obvious, severe, and very severe. Then the option was converted into a score for expression, where 1 point was for none, 2 points were for mild, 3 points were for obvious, 4 points were for severe, and 5 points were for very severe. A score before taking was then subtracted from a score after taking, and then the difference was divided by the score before taking, and the quotient was converted into a percentage.

### Test Mode:

The 8 subjects in the experimental group were allowed to consume 0.35 g of the Sparassia crispa water extract prepared in Example 1 daily for 4 consecutive weeks.

Data measured before consumption (i.e., week 0) was called the blank group, and data measured after 4 weeks of consumption (i.e., week 4) was called the experimental group.

### Test Results:

As shown in the figure below, on the basis of a mean of all the subjects and the data at week 0 (considered as 100% or 1), the relative value of each group was obtained by conversion. The standard deviation was calculated by using the STDEV formula of Excel software, whether there was a statistically significant difference or not was analyzed with a single-tailed student t-test in an Excel software to obtain its p-value. Moreover, in the figure, “*” represents that its p-value is less than 0.05.

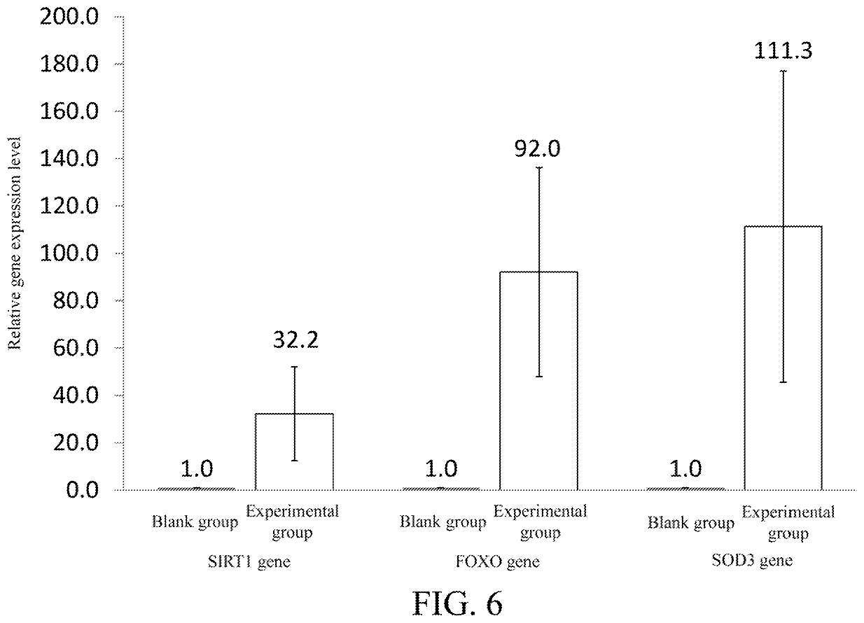

See FIG. 6. The subjects in the blank group did not consume the Sparassia crispa extract, which means that an average expression level was set as 1.0 under normal physiological metabolism conditions. The 3 of the 8 subjects were excluded due to the fact that errors in experimental data were caused by poor RNA extraction quality. Here, the 3 of the remaining 5 subjects were improved in their gene expression levels, which means that the proportion of improved individuals was 60%. An average SIRT1 gene expression level of these 5 subjects in the experimental group was increased by 32.2 times in comparison to that at week 0 (blank group) after 4 weeks of consumption of the Sparassia crispa extract (experimental group), an average FOXO gene expression level of these 5 subjects in the experimental group was increased by 92.0 times in comparison to that at week 0 (blank group) after 4 weeks of consumption of the Sparassia crispa extract (experimental group), and an average SOD3 gene expression level of these 5 subjects in the experimental group was increased by 111.3 times in comparison to that at week 0 (blank group) after 4 weeks of consumption of the Sparassia crispa extract (experimental group).

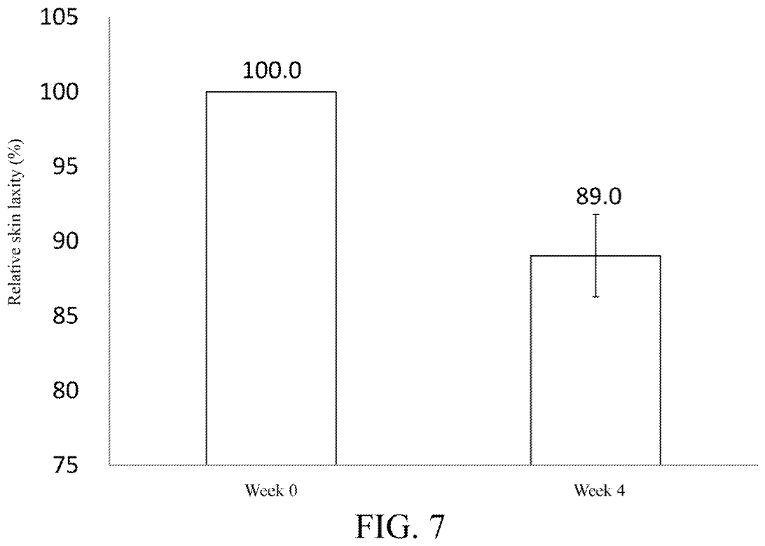

See FIG. 7. After taking the Sparassia crispa extract daily for 4 weeks, the 2 subjects were excluded due to the fact that the detection probe was blocked by severe desquamation of skin to result in errors in experimental data. Here, the skin laxity of 5 of the remaining 6 subjects was decreased, which means that the proportion of improved individuals was 83.3%. Average skin laxity of 6 subjects in the experimental group was decreased from 100% (blank group) at week 0 to 89.0% (experimental group). This means that the Sparassia crispa extract can help firm the skin.

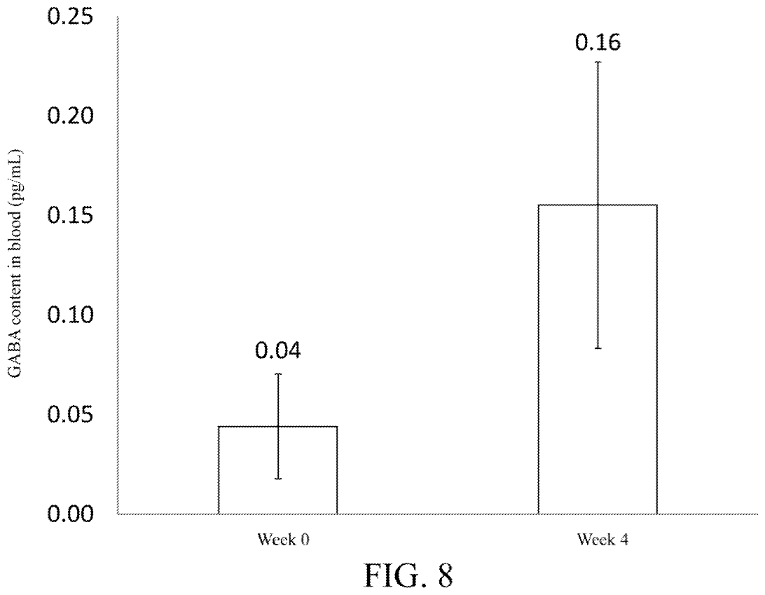

See FIG. 8. After taking the Sparassia crispa extract daily for 4 weeks, 6 of the 8 subjects showed an increase in the GABA content in their blood, which means that the proportion of improved individuals was 62.5%. The average GABA content in blood of 8 subjects in the experimental group was increased from 0.04 pg/mL (blank group) at week 0 to 0.16 pg/mL (experimental group), which means that it was increased by four times on average.

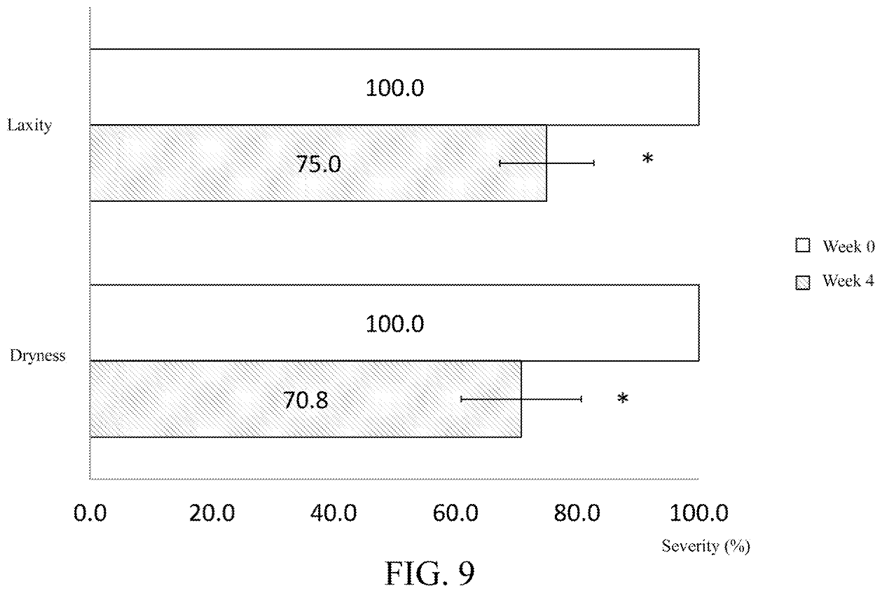

See FIG. 9. After taking the Sparassia crispa extract daily for 4 weeks, 6 of 8 subjects self-rated that the skin laxity was improved, which means that the proportion of improved individuals was 62.5%. Continue to see FIG. 9, in a case where self-rated scores of 8 subjects in the experimental group on the severity of the skin laxity were summed and a mean thereof was obtained, the mean was compared to a total score mean in an initial state (week 0), then was divided by the total score mean in the initial state, and the quotient was converted into a percentage, it can be obtained by conversion that the severity of the skin laxity was reduced by 25.0%.

After taking the Sparassia crispa extract daily for 4 weeks, 6 of the 8 subjects self-rated that the skin dryness was improved, which means that the proportion of improved individuals was 62.5%. Continue to see FIG. 9, in a case where self-rated scores of 8 subjects in the experimental group on the severity of the skin dryness were summed and a mean thereof was obtained, the mean was compared to a total score mean in an initial state (week 0), then was divided by the total score mean in the initial state, and the quotient was converted into a percentage, it can be obtained by conversion that the severity of the skin dryness was reduced by 29.2%.

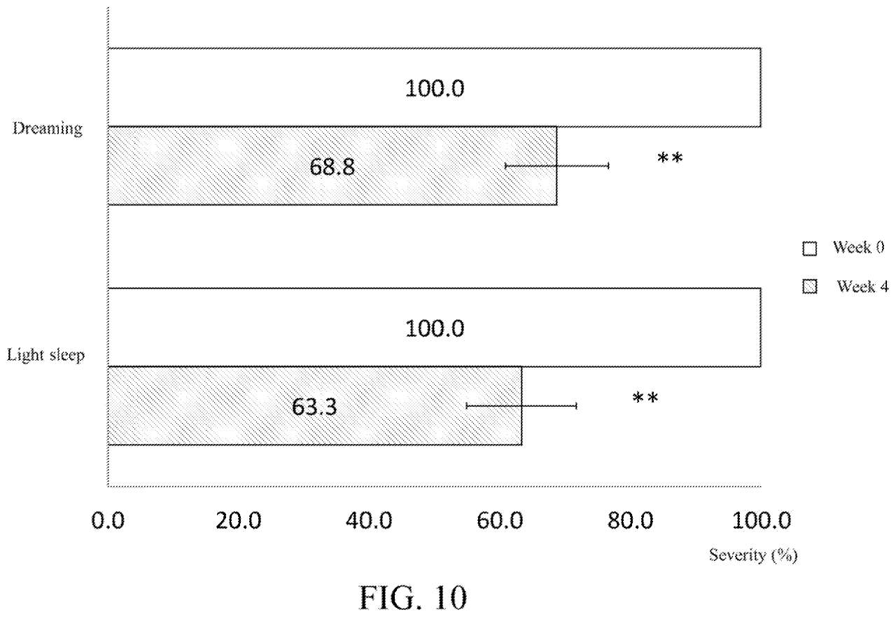

See FIG. 10. After taking the Sparassia crispa extract daily for 4 weeks, 6 of the 8 subjects self-rated that dreaming at night was reduced, which means that the proportion of improved individuals was 75.0%. Continue to see FIG. 10, in a case where self-rated scores of 8 subjects in the experimental group on the severity of the dreaming were summed and a mean thereof was obtained, the mean was compared to a total score mean in an initial state (week 0), then was divided by the total score mean in the initial state, and the quotient was converted into a percentage, it can be obtained by conversion that the severity of the dreaming at night was reduced by 31.3%.

After taking the Sparassia crispa extract daily for 4 weeks, 6 of the 8 subjects self-rated that the light sleep at night was reduced, which means that the proportion of improved individuals was 75.0%. Continue to see FIG. 10, in a case where self-rated scores of 8 subjects in the experimental group on the severity of the light sleep were summed and a mean thereof was obtained, the mean was compared to a total score mean in an initial state (week 0), then was divided by the total score mean in the initial state, and the quotient was converted into a percentage, it can be obtained by conversion that the severity of the light sleep was reduced by 36.7%.

In summary, the Sparassia crispa extract of the embodiments of the present disclosure can be used for preparing compositions for improving sleep quality, increasing mitochondrial expression, repairing cell damage, and firming the skin of the subject. In some embodiments, the Sparassia crispa extract has at least one of the following effects: increasing gamma-aminobutyric acid (GABA) content, contributing to nerve relaxation, enhancing activity of mitochondria in cells, extending cell lifespan, regulating autophagy of mitochondria in cells, regulating ATG1 gene, ATG8 gene, CCT5 gene, CCT7 gene, Pink1 gene, Parkin gene, SIRT1 gene, FOXO gene, PARP1 gene, and SOD3 gene of human cells, reducing laxity of the skin of the subject, and reducing dryness of the skin of the subject.

Although the present disclosure has been described in considerable detail with reference to certain preferred embodiments thereof, the disclosure is not for limiting the scope of the disclosure. Persons having ordinary skill in the art may make various modifications and changes without departing from the scope and spirit of the disclosure. Therefore, the scope of the appended claims should not be limited to the description of the preferred embodiments described above.

What is claimed is:

1. A method for reducing light sleep and/or dreaming in a subject in need thereof, comprising: administering to the subject an effective dose of the Sparassia crispa extract, wherein the Sparassia crispa extract is obtained by extracting Sparassia crispa by water.

2. The method according to claim 1, wherein the Sparassia crispa extract increases gamma-aminobutyric acid (GABA) content of the subject.

3. The method according to claim 1, wherein the Sparassia crispa extract contributes to nerve relaxation of the subject.

4. A method for increasing mitochondrial expression in a subject in need thereof, comprising: administering to the subject an effective dose of the Sparassia crispa extract, wherein the Sparassia crispa extract is obtained by extracting Sparassia crispa by water.

5. The method according to claim 4, wherein the Sparassia crispa extract enhances activity of mitochondria in cells of the subject.

6. The method according to claim 4, wherein the Sparassia crispa extract extends cell lifespan of the subject.

7. The method according to claim 4, wherein the Sparassia crispa extract regulates autophagy of mitochondria in cells of the subject.

8. The method according to claim 4, wherein the Sparassia crispa extract regulates an expression level of a mitochondrial expression-related gene in cells of the subject, and the mitochondrial expression-related gene is at least one of: a CCT5 gene, a CCT7 gene, a Pink1 gene, a Parkin gene, an SIRT1 gene, an FOXO gene, a PARP1 gene, and an SOD3 gene.

9. A method for repairing cell damage in a subject in need thereof, comprising: administering to the subject an effective dose of the Sparassia crispa extract, wherein the Sparassia crispa extract is obtained by extracting Sparassia crispa by water.

10. The method according to claim 9, wherein the Sparassia crispa extract regulates autophagy of cells of the subject.

11. The method according to claim 9, wherein the Sparassia crispa extract enhances an expression level of an ATG1 gene and/or an ATG8 gene of the subject.

12. The method according to claim 9, wherein the Sparassia crispa extract increases gamma-aminobutyric acid content of the subject.

13. A method for firming skin of a subject in need thereof, comprising: administering to the subject an effective dose of the Sparassia crispa extract, wherein the Sparassia crispa extract is obtained by extracting Sparassia crispa by water.

14. The method according to claim 13, wherein the Sparassia crispa extract reduces laxity of the skin of the subject.

15. The method according to claim 13, wherein the Sparassia crispa extract reduces dryness of the skin of the subject.

16. The method according to claim 13, wherein the Sparassia crispa extract is obtained by extracting a fruiting body of the Sparassia crispa.

17. The method according to claim 13, wherein the Sparassia crispa extract is obtained by extracting the Sparassia crispa in water at 90° C.±5° C. for 60 mins.


SEQUENCE LISTING (20 sequences)

SEQ ID NO: 1 - DNA, 20 bp, synthetic construct
  source 1..20: mol_type = other DNA
cggataagtgccccacctta

SEQ ID NO: 2 - DNA, 17 bp, synthetic construct
  source 1..17: mol_type = other DNA
tccagtgcgtcggcaaa

SEQ ID NO: 3 - DNA, 24 bp, synthetic construct
  source 1..24: mol_type = other DNA
gtggcatggacaagcttattgtag

SEQ ID NO: 4 - DNA, 19 bp, synthetic construct
  source 1..19: mol_type = other DNA
cagaattgtggccccatca

SEQ ID NO: 5 - DNA, 20 bp, synthetic construct
  source 1..20: mol_type = other DNA
ctgtggtggctagtgctcct

SEQ ID NO: 6 - DNA, 20 bp, synthetic construct
  source 1..20: mol_type = other DNA
tccagacgtgagacagttgg

SEQ ID NO: 7 - DNA, 20 bp, synthetic construct
  source 1..20: mol_type = other DNA
gcagagaccgtggagaaaag

SEQ ID NO: 8 - DNA, 20 bp, synthetic construct
  source 1..20: mol_type = other DNA
cttttctccacggtctctgc

SEQ ID NO: 9 - DNA, 23 bp, synthetic construct
  source 1..23: mol_type = other DNA
caggaggacgagaacacggtgtc

SEQ ID NO: 10 - DNA, 23 bp, synthetic construct
  source 1..23: mol_type = other DNA
ggaaggttctttggcaccagcac

SEQ ID NO: 11 - DNA, 21 bp, synthetic construct
  source 1..21: mol_type = other DNA
tatccagaccgtgtgcccgtc

SEQ ID NO: 12 - DNA, 22 bp, synthetic construct
  source 1..22: mol_type = other DNA
gtggatgcgcttgcgaatgagg

SEQ ID NO: 13 - DNA, 23 bp, synthetic construct
  source 1..23: mol_type = other DNA
tagccttgtcagataaggaagga

SEQ ID NO: 14 - DNA, 22 bp, synthetic construct
  source 1..22: mol_type = other DNA
acagcttcacagtcaactttgt

SEQ ID NO: 15 - DNA, 19 bp, synthetic construct
  source 1..19: mol_type = other DNA
cggacaaacggctcactct

SEQ ID NO: 16 - DNA, 21 bp, synthetic construct
  source 1..21: mol_type = other DNA
ggacccgcatgaatcgactat

SEQ ID NO: 17 - DNA, 20 bp, synthetic construct
  source 1..20: mol_type = other DNA
agcgtgtttctaggtcgtgg

SEQ ID NO: 18 - DNA, 20 bp, synthetic construct
  source 1..20: mol_type = other DNA
catcaaacatgggcgactgc

SEQ ID NO: 19 - DNA, 18 bp, synthetic construct
  source 1..18: mol_type = other DNA
agctggaaaggtgcccga

SEQ ID NO: 20 - DNA, 21 bp, synthetic construct
  source 1..21: mol_type = other DNA
cttggcgtacatgtctcggat